(19) United States
(12) Patent Application Publication
(10) Pub. No.: US 2019/0053469 A1
(43) Pub. Date: Feb. 21, 2019

(54) DOG TEMPERATURE SYSTEM

(71) Applicant: Matthew A. MARDIROSSIAN, Potomac, MD (US)

(72) Inventor: Matthew A. MARDIROSSIAN, Potomac, MD (US)

(21) Appl. No.: 15/680,342

(22) Filed: Aug. 18, 2017

(51) Int. Cl. A01K 27/00; G01K 1/02; G01K 13/00; A61B 5/00

(52) U.S. Cl. CPC A01K 27/009; A01K 27/001; G01K 1/024; A61B 5/01; G01K 13/002; A61B 5/0008; G01K 1/026

(57) ABSTRACT

A fever detection system includes a temperature sensor; and a dog collar including a power source, an output device, and a processing circuit coupled to the power source and the output device. The processing circuit is configured to: periodically receive temperature measurements from the temperature sensor, based on the received temperature measurements, determine whether the received temperature measurements exceed a set temperature limit, and upon determining that the received temperature measurements exceed the set temperature limit, transmit a signal to the output device to activate a visual and/or auditory output indicating detection of a fever (including overheating).

### FIELD OF THE INVENTION

This invention relates generally to methods and systems for determining health condition, more particularly, to detecting health condition of a pet based on pet's temperature measurements and providing a notification to a pet owner or other(s). This invention also relates to monitoring health condition of humans (e.g., children) using one or more sensors and providing a location and/or remote notifications when the health condition reaches undesired levels.

### BACKGROUND AND SUMMARY OF THE INVENTION

Fever is typically used to describe elevated body temperature caused by infection or inflammation. A normal body temperature for dogs is between 101 and 102.5 F. A temperature of more than 103 F in a dog can be considered a fever. When temperature reach 106 F, fatal complications can occur in the dog. Temperature of a dog may increase also due to high external temperatures or exercise. Accordingly, it is very important to know a dog's temperature and when this temperature exceeds normal levels.

However, in practice a dog's temperate is taken by the pet owner or a veterinarian only when other symptoms indicate that there may be a problem. Such symptoms may include, lethargy, depressed mood, shivering, loss of appetite, vomiting, coughing, or nasal discharge. Because these symptoms can be missed or there is a delay in detecting them, there is a need for systems and methods which can detect a fever quickly and accurately.

Dog owners are told that you can tell whether your dog has a fever by feeling his nose. If the nose is wet and cold, then there is no fever. If the nose is hot and dry, he probably has a fever. While this method may provide some insight dog's condition, the nose test alone is not enough for an accurate assessment of the presence of a fever.

More accurate results for detecting the presence of a fever can be obtained by measuring the dog's temperature rectally. While more accurate, this approach is invasive, requires for the dog to remain still, and may require more than one person to complete. In addition, rectally measuring the temperature is time consuming because it is recommended to hold the thermometer in place for at least two minutes.

Ear thermometers provide a less invasive technique to measure a dog's temperate because they measure heat that is emitted from the dog ear. However, using an ear thermometer also requires for the dog to remain still. In addition, accuracy of the temperature measurements using the ear thermometer greatly depend on the proper use of the ear thermometer.

The challenges of monitoring a dog's health condition is also faced with other pets and even people. For example, the health condition of a child or pet (e.g., dog) in another part of the house or another remote location (e.g., in a car) may need to be known but not accessible.

Certain example embodiments of the instant invention provide solution(s) to easily and accurately detect health condition of pets and people. One specific example provides for detecting the presence of a fever (including overheating) in a dog. Example embodiments provide for health condition to be monitored based on temperature measurements, heart rate measurements, and/or breathing monitors and to provide a visual and/or auditory notifications when the health condition reached undesirable levels. As discussed in more detail below, the visual or auditory indication can be provided via a dog collar, arm band, or ankle band. In addition, a notification may be sent to remote location, first aid responder, and/or a mobile device.

In example embodiment of this invention, there is provided a fever detection system comprising a temperature sensor; and a dog collar including a power source, an output device, and a processing circuit coupled to the power source and the output device. The processing circuit is configured to: periodically receive temperature measurements from the temperature sensor; based on the received temperature measurements, determine whether the received temperature measurements exceed a set temperature limit; and upon determining that the received temperature measurements exceed the set temperature limit, transmit a signal to the output device to activate a visual and/or auditory output indicating detection of a fever.

In another example embodiment of this invention, there is provided a dog collar comprising: a temperature sensor, an output device configured to output a visual output and/or sound; and a processing circuit coupled to the temperature sensor and the output device. The processing circuit is configured to: receive temperature measurements from the temperature sensor, based on the received temperature measurements, determine whether the received temperature measurements indicate a fever, and, upon determining that the received temperature measurements indicate a fever, control the output device to output a visual output and/or sound indicating detection of the fever. The temperature sensor, the output device, and the processing circuit are commonly housed.

In another example embodiment of this invention, there is provided a method for detecting a fever in a dog, the method comprising: receiving temperature measurements from a temperature sensor included in a collar, based on the received temperature measurements, determine whether the received temperature measurements exceed a set temperature limit, and upon determining that the received temperature measurements exceed the set temperature limit, activate a visual and/or auditory output on the collar to indicate detection of a fever.

In another example embodiment of this invention, there is provided a wearable device comprising: a temperature sensor, a heart rate sensor, an output device including a strobe light or high frequency noise generator, and a processing circuit coupled to the temperature sensor and the output device. The processing circuit is configured to: receive temperature measurements from the temperature sensor; receive heart rate measurements from the heart rate sensor; based on the received temperature measurements and/or heart rate measurements, determine whether the received temperature and/or heart rate measurements exceed set limits; and upon determining that the received temperature and/or heart rate measurements exceed set limits, control the output device to output a visual output using the strobe light and/or a sound using the high frequency noise generator.

In another example embodiment of this invention, there is provided a dog collar comprising: a temperature sensor, communication circuitry, an output device configured to output a visual output and/or sound, and a processing circuit. The processing circuit is coupled to the temperature sensor, the output device, and the communication circuitry. The processing circuit is configured to: receive temperature measurements from the temperature sensor; based on the received temperature measurements, determine whether the received temperature measurements exceed a first limit; and based on the received temperature measurements, determine whether the received temperature measurements exceed a second limit higher than the first limit. Upon determining that the received temperature measurements exceed the first limit, processing circuit controls the output device to output a first visual output and/or a second sound. Upon determining that the received temperature measurements exceed the second limit, (1) control the output device to output a second visual output and/or a second sound, and (2) transmit, via the communication circuitry, a notification to a mobile device.

### BRIEF DESCRIPTION OF THE DRAWINGS

These and other features and advantages may be better and more completely understood by reference to the following detailed description of exemplary illustrative embodiments in conjunction with the drawings, of which:

FIG. 1 illustrates health detection system according to an embodiment of the present disclosure;

FIG. 2 illustrates a method for detecting health conditions according to an embodiment of the present disclosure;

FIG. 3 illustrates wearable device according to an embodiment of this disclosure; and

FIG. 4 illustrates a method for detecting health conditions according to an embodiment of the present disclosure.

### DETAILED DESCRIPTION

This invention relates to measuring health condition of pets and/or people and providing notification(s) when the health condition reaches undesirable levels. In one example, this invention relates to preventative measures regarding the overheating of animals and can be extended to humans. In one example, the exemplary embodiments relate to detecting a fever in pet (e.g., a dog) and providing an indication of the fever to a pet owner. The temperature monitoring may be related to walking dogs in very hot weather, and dogs or children left in cars or overheating at sports event. In another example, this invention relates to an arm or ankle band for humans which monitors breathing sequence as related to heartbeat and temperature.

Certain example embodiments of the instant invention provide an automated method to accurately detect a fever in a pet and provide a notification upon detecting the fever. Example embodiments provide for periodic monitoring of a pet's temperature and using the detected temperature to determine whether the pet has a temperature exceeding a preset limit. Temperatures exceeding the preset limit may indicate that the pet has a fever. The proposed methods and systems provide for a less intrusive approach as compared to traditional methods for measuring a pet's temperature, improved accuracy due to the continued periodic monitoring and reduction in erroneous temperature readings, faster detection of the fever, and less involvement of the pet owner.

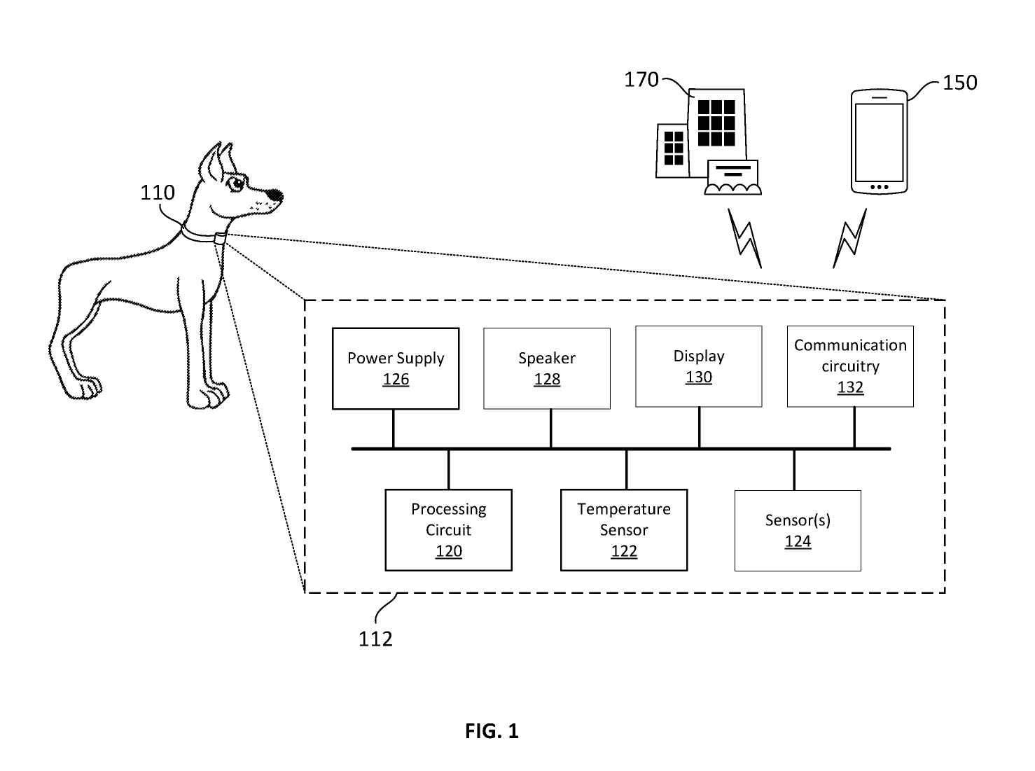

FIG. 1 illustrate a fever detection system according to an embodiment of the present disclosure. The fever detection system includes a collar 110 which may be put on a dog or other pet. The monitoring equipment described with reference to FIG. 1 may be provided in a prong collar. The collar 110 may include one or more housings 112, provided on the collar 110. The one or more housings 112 may include components for measuring the temperature of the dog and determining whether the measured temperature indicates a fever or other undesirable condition of the dog.

As illustrated in FIG. 1, the housing 112 may include a processing circuit 120, a temperature sensor 122, one or more sensors 124, a power supply 126, a speaker 128, a display 130, and communication circuitry 132. The components of the fever detection system may be communicatively coupled to one or more other components of the fever detection system. For example, the temperature sensor 122, the one or more sensors 124, the power supply 126, the speaker 128, the display 130, and the communication circuitry 132 may each be directly coupled to the processing circuit 120.

The processing circuit 120 may include one or more modules configured to perform the various operations disclosed in this application. The processing circuit 120 may include one or more processors and memory configured to receive data from the temperature sensor 122, the one or more sensors 124, and/or the communication circuitry 132, process the data, and transmit data to the speaker 128, the display 130, and the communication circuitry 132. In some embodiments, the processing circuit 120 may transmit requests for data to the temperature sensor 122 and/or the one or more other sensors 124 periodically or based on detection of certain event.

The memory in the processing circuit 120 may store program instructions for processing received data and may store received data. The program instructions may provide instructions for analyzing the received data, and instructions for performing operations when predetermined conditions are detected. For example, the program instructions may provide instructions for analyzing the received temperature data from the temperature sensor 122 and instructions for sending signals to the speaker 128 and/or the display 130 when the received temperature data is determined to exceed preset limits.

The information stored in the memory may include dog specific information such as acceptable temperatures, dog identification information, pet owner identification information, temperature history, and conditions under which the dog is to be determined to have a fever or in bad health.

The temperature sensor 122 may be configured to take a temperature of the dog. The temperature sensor 122 may include a skin temperature sensor, infrared thermometer, and/or mini or micro thermistors. The temperature sensor 122 may be provided in a housing on the collar. In other embodiments, the temperature sensor 122 may be provided outside of the housing (e.g., other portions of the collar or away from the collar) and communicate with the processing circuit 120 via a wired or wireless connection. A plurality of temperature sensors 122 may be provided at different locations on the dog. For example, a plurality of temperature sensors may be disposed along different locations on the collar 110. In other embodiment, the temperature sensors 122 may be attached to patches or bands that are attached to other parts of the dog.

The one or more sensors 124 may include other sensors such as external temperature sensors, heart rate sensors, and motion sensors. The external temperature sensor may measure the air temperature. The heart rate sensor may measure the dog's heart rate. The motion sensor may measure linear acceleration and/or angular rate of rotation about one or more axes. The data from these sensors may be processed by the processing circuit 120 to make more meaningful decisions as to whether the dog has a fever or is in bad health.

The power supply 126 may provide power to the processing circuit 120 and other components in the collar. The power supply 126 may be rechargeable and replaceable.

The speaker 128 may be configured to output sound responsive to instructions. The speaker 128 may receive a sound single from the processing circuit 120 to be played or may store a sound signal and output the stored sound signal responsive to instructions from the processor.

The display 130 may include one or more lights (e.g., LEDs) or an LCD displays. The display 130 may be configured to provide visual output responsive to instructions from the processing circuit 120. For example, a first light (e.g., a red LED) may be activated to indicate that the measured temperatures exceed a preset limit and that the dog may have a fever. The display 130 may include a second light (e.g., a green LED) to indicate that the measured temperature does not exceed a preset limit. The second light may provide an indication to the pet owner that the system is operating properly. In one example, a notification (e.g., using text) may be displayed on a LCD indicating that fever is detected. The display 130 may be configured to display an estimated temperature of the dog, and/or change color of the display to indicate level of temperature.

The communication circuitry 132 may be configured to wirelessly communicate with one or more mobile devices 150, one or more communication devices provided in other locations 170, and/or one or more sensors provided outside of the collar 110. The communication circuitry 132 may connect the components in the collar 110 to the mobile device(s), sensor(s), computers at other locations 170 by utilizing Bluetooth technology, a wireless network, or network utilizing 3G/4G/5G/CDMA/LTE. The mobile device 150 may be a smartphone, PDA, cellphone, smart glasses, tablet device, pad device, or another portable electronic device. The mobile device 122 may be a wearable device such as a watch, bracelet, activity tracker, a health monitoring device, or other wearable devices. The one or more communication device provided in other locations 170 may include a computer provided at a pet owners work location, veterinary physician, and/or person providing monitoring of pets health.

The communication circuitry 132 may provide information about the dog's health to the one or more mobile devices 150 and one or more communication devices provided in other locations 170. As discussed in more detail below, a first indication may be provided via the collar 110 when a temperature of the dog exceeds a first limit and a second indication may be provided on the collar 110 and/or sent to the mobile devices 150 and/or one or more communication devices provided in other locations 170.

While the discussion of the system illustrated in FIG. 1 is made with reference to a pet collar, the system may be provided as part of an arm or ankle band for humans which monitors the human's health condition (e.g., their breathing sequence as related to heartbeat and temperature).

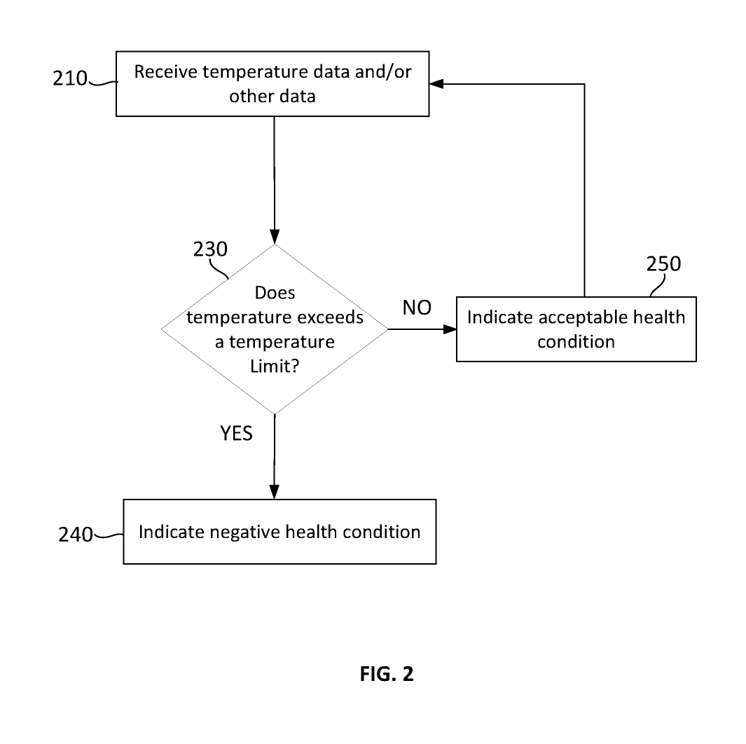

FIG. 2 illustrates a method for detecting health condition according to an embodiment of the present disclosure. The method include receiving temperature data from one or more temperature sensors 210. Data may also optionally be received from other sensors (e.g., air temperature sensor, motion sensor, heart rate sensor). The data may be received periodically or continuously. In some embodiment, the data may be received in response to a trigger requesting the data.

The received data is analyzed to determine if the received temperature data and/or other sensor data exceed preset limits 230. For example, the determination may be made as to whether the temperature measured by the temperature sensor exceeds a preset temperature limit. In a specific example, the determination may be made as to whether the temperature measured by the temperature sensor exceeds 103 F. The temperature limit may be a preset value. The temperature limit may be set based on the type of dog for which the temperature is being measured. Similar determinations may be made for data received from other sensors (e.g., air temperature sensor, motion sensor, heart rate sensor). If the values from the other sensors exceed limits for each different sensor, a determination is made the health condition of the pet or person is not acceptable.

In some embodiments, the temperature limit may be set by a user activating an input to calibrate the temperature limit. This may be performed when the fever detection system is first put on the dog. This process may establish a baseline of an acceptable temperature and the limit may be set by adding a predetermined value (e.g., two degrees F.) to the acceptable temperature.

In one embodiment, the temperature limit may be adjusted based on data received from other sensors (e.g., motion sensor, heart rate sensor, and/or air temperature sensor). For example, if the dog is running the temperature of the dog will rise. Accordingly, when the motion sensor indicates that there is motion exceeding a predetermined motion for at least a predetermined period of time, when the heart rate increases for a predetermined period of time, and/or when the air temperature sensor indicated that the outside temperature is high, the temperature limit may be adjusted (e.g., increased by 0.5 or 1 degrees F.).

If the measured temperature exceeds the set limit (YES in step 230), then the determination may be made that negative health condition is present (e.g., fever is detected). If the measured temperature does not exceed the set limit (NO in step 230), then the determination may be made that health condition is normal (e.g., fever is not detected).

If the fever is negative health condition is detected (YES in step 230), then it is indicted that negative heath condition is detected in step 240. Indicating that the negative heath condition is detected may include activating a light, outputting sound with an indication that the negative heath condition is detected, and/or displaying text (e.g., on an LCD, a collar or via a mobile device and/or a computer at another location). In some embodiments, indicating that negative heath condition is detected may include sending a notification (e.g., text message or email) to a registered owner or caregiver of the dog.

If the negative heath condition is not detected (NO in step 230), then it is indicted that fever is not detected in step 240. Indicating that the negative heath condition is not detected may include deactivating a light and/or displaying text (e.g., on an LCD on a collar or via a mobile device). In certain example embodiments, a second light may be activated when it is determined that negative heath condition is not detected.

In certain example embodiments, indicating that the negative heath condition is detected may also include an indication of the temperature level and/or heart rate via text on an LCD or activation of an array of lights. For example, the display may include an array of LEDs and the number of activated LED may correspond to the level of the measured temperature and/or heart rate. In another example, the color of the LED may change based on the level of the detected temperature and/or heart rate.

In certain example embodiments, indicating that the negative heath condition is detected may include providing a first indication if the measured temperate exceeds a first temperature limit (e.g., 103 F) and providing a second indication if the measured temperate exceeds a second temperature limit (e.g., 106 F). The first indication may correspond to activating an LED, and the second indication may include providing an auditory indication via a speaker and/or sending a notification to the mobile device or a nearest emergency operator with communications office. A GPS located in the wearable device may provide information as to the location of the wearable device when the notification.

As known to a person of ordinary skill in the art a temperature detected at the outside surface of a dog's body or human body may not represent the actual internal temperature. While the temperature detected at the outside surface of a body may not strictly represent the internal temperature, changes in temperature of the outside will correspond to changes in the dog's internal temperature and may be used to detect a negative heath condition. Accordingly, significant changes in the outside body temperature (e.g., changes exceeding a present value) will indicate that the pet or person likely has the poor health condition (e.g., a fever).

According to an exemplary embodiment, an output signal is recorded and compared to the standard rerecording over time. If the output signal is above standard levels, the signal will synch and be sent to the owner's phone or nearest emergency operator with communications office. The location (GPS) information can be transmitted with the all the data. The transmitted data may include identification information of the pet or person.

According to an exemplary embodiment, a strobe light or high frequency noise generator may be included and activated to alert individual in approximate to the pet or human in distress.

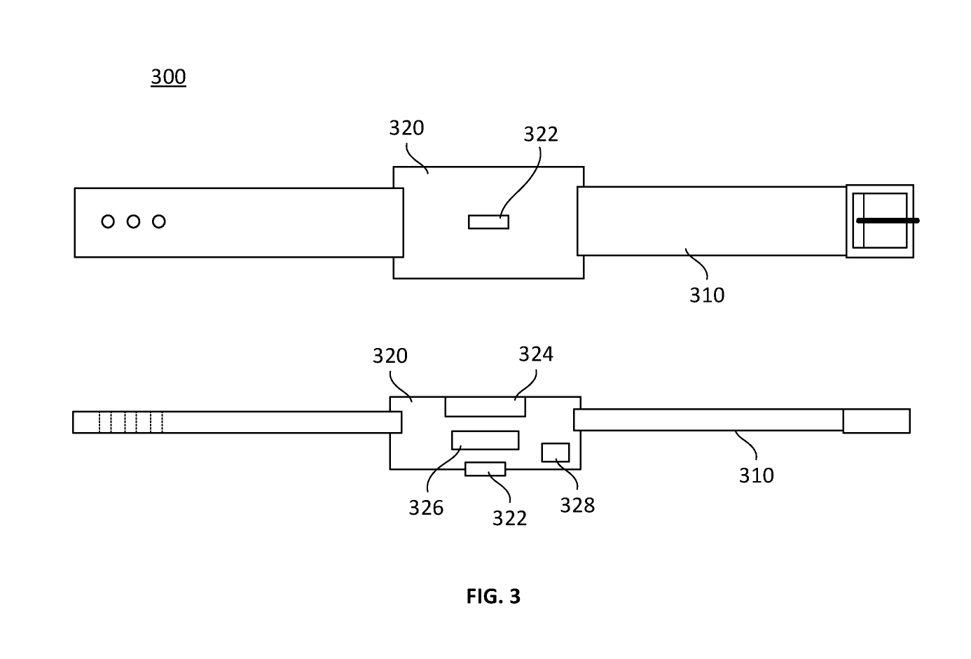

FIG. 3 illustrates a top and side views of a wearable device 300 according to an embodiment of this disclosure. The wearable device 300 may be a pet collar (e.g., a dog prong collar) or an arm or ankle band. The wearable device may include a strap 310 and a housing 320 attached to the strap 310. The position of the housing 320 on the strap 310 may be adjustable.

The housing 320 may include components for measuring one or more parameters of a pet or human (e.g., a dog's temperature), comparing the measured temperature to a set limits, and provide a visual or auditory indication of the results of the comparison. As illustrated in FIG. 3, the housing 320 may include an indicator 322, a temperature sensor 324, a processing circuit 326, and a battery 328.

The indicator 322 may include an LED that is configured to light up when the processing circuit 326 determines based on the received temperature measurements that the temperature exceed a set limit. While a single indicator 322 is illustrated, an array of indicators may be arranged along the housing 320 and/or the strap 310.

The temperature sensor 324 may be provided on the surface of the housing that is opposite to the surface including the indicator. The temperature sensor 324 may be provided such that the temperature is adjacent to the pets or humans body surface when the wearable device is worn.

In one embodiment, the processing circuit may include a comparator to compare a voltage or current, representing the measured temperature, provided form the temperature sensor to a preset voltage or current. The comparator may activate an LED when the voltage or current, representing the measured temperature, exceeds the preset voltage or current. This embodiments provides a compact and inexpensive configuration to provide a detection of the temperature and indication when the temperature reaches unacceptable values.

The housing 320 may include other components (e.g., see components illustrated in FIG. 1) not illustrated in FIG. 3. In addition, a plurality of housings may be provided along the strap 310. In one embodiment, the temperature sensor 324 may be provided in a different housing from the indicator 322.

One or more other components illustrated in FIG. 1 may be included in the housing. For example, other sensors (e.g., heartbeat sensor) and wireless communication circuitry may be included in the housing.

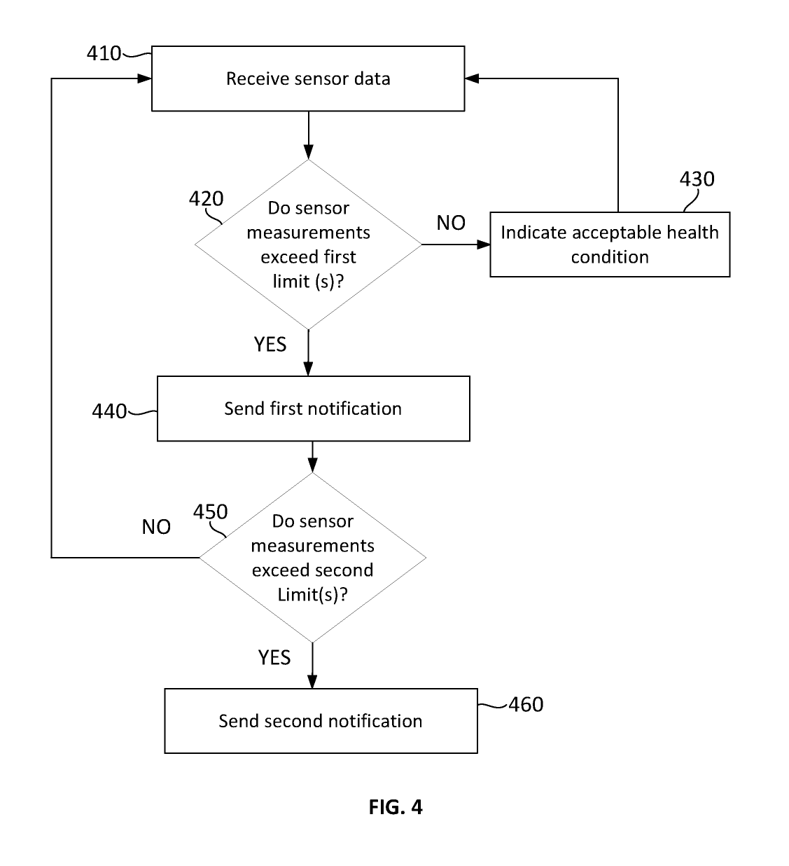

FIG. 4 illustrates a method for detecting health conditions according to another embodiment of the present disclosure. The method illustrated in FIG. 4 provides an example of sending multiple notification when one or more measured parameters (e.g., the temperature and/or the heart rate) exceed preset limit(s).

The method include receiving sensor data from one or more sensors 410. The sensor data may include data from an air temperature sensor, a motion sensor, and/or a heart rate sensor. The data may be received periodically or continuously. In some embodiment, the data may be received in response to a trigger requesting the data.

The received sensor data is analyzed to determine if the received sensor measurements exceed a first limit 420. For example, the determination may be made as to whether the temperature measured by the temperature sensor exceeds a set first limit (e.g., 103 F).

If the measured sensor data does not exceed the first limit (NO in step 420), then the process may display an indication that the health condition is acceptable (e.g., temperature is OK) 430 and/or return to receiving additional data 410.

If the measured temperature exceeds the first limit (YES in step 420), a first notification may be sent 440 to indicate high temperature reading. The first notification may include by lighting a light on the wearable device, and/or sending a notification to a registered mobile device. The determination in step 420 may be performed for one or more sensors, with each type of sensor data being compared to respective first set of limits. In one embodiment, if at least one of the sensors exceeds the set limit, then the determination may be made the sensor measurements exceed the first limit. In another embodiment, the sensor data from each sensor will need to exceed the respective limit before the determination is made that the sensor measurements exceed the first limit.

In step 450, the received data is analyzed to determine if the received sensor data from one or more sensors exceed second limit(s). For example, the determination may be made as to whether the temperature measured by the temperature sensor exceeds a set second limit (e.g., 106 F). If the measured sensor data does not exceed the second limit(s) (NO in step 450), then the process may return to receiving additional data 410.

If the measured sensor data exceeds the second limit(s) (YES in step 450), a second notification may be sent 460 to indicate high temperature reading. The second notification may include lighting a second light on the collar, notifying nearest emergency operator, and/or sending a notification to a mobile device and/or another remote location (e.g., veterinarian's office).

The determination in step 450 may be performed for one or more sensors, with each type of sensor data being compared to respective second set of limits. In one embodiment, if at least one of the sensors exceeds the set limit, then the determination may be made the sensor measurements exceed the second limit. In another embodiment, the sensor data from each sensor will need to exceed the respective limit before the determination is made that the sensor measurements exceed the second limit.

While the example embodiment disclosed in this application are discussed with reference to a dog, embodiment of this disclosure are not so limited and may be applied to other pets and humans wearing a wearable device.

Note that “fever” as used herein includes overheating, and does not require illness or sickness. Thus, for example, if a dog overheats because the dog is trapped in a hot car with the windows up, this would be covered by “fever” even though the dog does not have a bacterial infection or viral illness.

Also, in the description and claims, the terms “coupled” and “connected,” along with their derivatives, may be used. In some embodiments of the invention, “connected” may be used to indicate that two or more elements are in direct physical or electrical contact with each other. “Coupled” may mean that two or more elements are in direct physical or electrical contact. However, “coupled” may also mean that two or more elements may not be in direct contact with each other, but may still cooperate or interact with each other.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered as examples because many other architectures can be implemented to achieve the same functionality.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. These software modules may configure a computing system to perform one or more of the example embodiments disclosed herein. Various functions described herein may be provided through a remote desktop environment or any other cloud-based computing environment.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as may be suited to the particular use contemplated.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A fever detection system comprising:
a temperature sensor; and
a collar including a power source, an output device, and a processing circuit coupled to the power source and the output device, the processing circuit configured to:
periodically receive temperature measurements from the temperature sensor;
based on the received temperature measurements, determine whether the received temperature measurements exceed a set temperature limit; and
upon determining that the received temperature measurements exceed the set temperature limit, transmit a signal to the output device to activate a visual and/or auditory output indicating detection of a fever.

2. The fever detection system of claim 1, wherein the temperature sensor is configured to wirelessly transmit the temperature measurements to the processing circuit.

3. The fever detection system of claim 1, wherein the power source, the output device, the processing circuit, and the temperature sensor are commonly housed and the housing is attached to the collar.

4. The fever detection system of claim 1, wherein the temperature sensor is separately housed from a housing including the power source, the output device, and the processing circuit.

5. The fever detection system of claim 1, wherein:
the temperature sensor is provided in a first housing attached to the collar;
the power source, the output device, and the processing circuit are provided in a second housing; and
the first housing is provided in a different location on the collar from the location of the second housing.

6. The fever detection system of claim 6, wherein the collar is a prong collar and the position of the first housing and/or the second housing on the collar is adjustable.

7. The fever detection system of claim 1, wherein the output device is an LED and the output device turns on the LED to indicate detection of the fever.

8. The fever detection system of claim 1, wherein the output device includes a first light that is activated when the received temperature measurements does not exceed the set temperature limit, and a second light that is activated when the received temperature measurements exceed the set temperature limit.

9. The fever detection system of claim 1, wherein the output device is a strobe light or high frequency noise generator.

10. The fever detection system of claim 1, wherein the processing circuit is further configured to, upon determining that the received temperature measurements exceed the set temperature limit, wirelessly transmit, to a mobile device, data indicating detection of the fever.

11. The fever detection system of claim 1, wherein the processing circuit is further configured to: based on the received temperature measurements over a period of time adjust the set temperature limit.

12. A wearable device comprising:
a temperature sensor;
a heart rate sensor;
an output device including a strobe light or high frequency noise generator; and
a processing circuit coupled to the temperature sensor and the output device, the processing circuit configured to:
receive temperature measurements from the temperature sensor;
receive heart rate measurements from the heart rate sensor;
based on the received temperature measurements and/or heart rate measurements, determine whether the received temperature and/or heart rate measurements exceed set limits; and
upon determining that the received temperature and/or heart rate measurements exceed set limits, control the output device to output a visual output using the strobe light and/or a sound using the high frequency noise generator.

13. The wearable device of claim 12, wherein the wearable device is one of a prong animal collar, arm band, or ankle band.

14. The wearable device of claim 12, wherein one or more of the set limits are determined based on recording standard recording of the temperature sensor and/or heart rate sensor over time.

15. The wearable device of claim 13, wherein upon determining that the received temperature and/or heart rate measurements exceed set limits a notification is sent to a mobile device or nearest emergency operator with communications office.

16. The wearable device of claim 15, wherein the notification includes location information.

17. A method for detecting a fever in a dog, the method comprising:
receiving temperature measurements from a temperature sensor included in a collar;
based on the received temperature measurements, determining whether the received temperature measurements exceed a set temperature limit;
upon determining that the received temperature measurements exceed the set temperature limit, activating a visual and/or auditory output on the collar to indicate detection of a fever.

18. The method of claim 17, further comprising, upon determining that the received temperature measurements exceed set temperature limit, wirelessly transmitting, to a mobile device, data indicating detection of the fever.

19. The method of claim 17, further comprising, based on the received temperature measurements over a period of time, adjusting the set temperature limit.

20. A dog collar comprising:
a temperature sensor;
communication circuitry;
an output device configured to output a visual output and/or sound; and
a processing circuit coupled to the temperature sensor, the output device, and the communication circuitry, the processing circuit configured to:
receive temperature measurements from the temperature sensor;
based on the received temperature measurements, determine whether the received temperature measurements exceed a first limit;
based on the received temperature measurements, determine whether the received temperature measurements exceed a second limit higher than the first limit;
upon determining that the received temperature measurements exceed the second limit, control the output device to output a first visual output and/or a first sound; and
upon determining that the received temperature measurements exceed the second limit, (1) control the output device to output a second visual output and/or a second sound, and (2) transmit, via the communication circuitry, a notification to a mobile device.
